(19) United States
(12) Patent Application Publication
(10) Pub. No.: US 2025/0308699 A1
(43) Pub. Date: Oct. 2, 2025

(54) SYSTEM AND METHOD FOR ASSESSING RISK PREDISPOSITION TO GESTATIONAL DIABETES BASED ON METHYLATION MARKERS, WEARABLES, AND SURVEY DATA

(71) Applicant: Ali Mostashari, New York, NY (US)

(72) Inventors: Ali Mostashari, New York, NY (US); Mario Storga, New York, NY (US); Klemo Vladimir, New York, NY (US); Marija Majda Perisic, New York, NY (US); Raya Khanin, New York, NY (US)

(73) Assignee: LifeNome Inc., New York, NY (US)

(21) Appl. No.: 18/616,506

(22) Filed: Mar. 26, 2024

(51) Int. Cl. G16H 50/30; G16B 20/20; G16B 40/20; G16H 50/20

(52) U.S. Cl. CPC G16H 50/30; G16B 20/20; G16B 40/20; G16H 50/20

(57) ABSTRACT

A method for computing predisposition risk for gestational diabetes mellitus (GDM) of an individual female based at least on methylation is provided. The method comprises receiving, by a computing device, methylation data including methylation markers for a female human. The method also comprises receiving, by the computing device, wearables data for the female. The method also comprises receiving, by the computing device, survey data provided by the female. The method also comprises applying, by the computing device, a risk predisposition predictor model to at least the received data to compute a risk predisposition to gestational diabetes mellitus of the female. The method also comprises the computer identifying methylation markers (CpGs) causally linked to gestational diabetes mellitus in the methylation data.

### GOVERNMENT SUPPORT STATEMENT

The invention was made by an agency of the United States Government or under a contract with an agency of the United States Government.

### FIELD OF THE DISCLOSURE

The present disclosure is in the field of assessing risk predisposition to gestational diabetes mellitus (GDM) during preconception or early pregnancy using epigenetic markers such as the methylation status of nucleotides (CpGs) in the genomic DNA from a biological sample. More particularly, the present disclosure provides systems and methods for identifying CpG markers causally linked to GDM, utilizing these markers in developing an accurate risk predictor of GDM using machine learning and computing a risk predisposition assessment of a subject individual woman, in many embodiments based on a platform that integrates methylation data, self-reported data, and wearables data to support computations of the risk predisposition.

### BACKGROUND

Gestational diabetes mellitus (GDM) is a widespread pregnancy complication with adverse implications for both maternal and offspring health. It is characterized by the onset of elevated blood sugar levels during pregnancy, typically occurring in the second trimester. GDM stands out as the most prevalent metabolic complication during pregnancy on a global scale. Criteria for diagnosis vary across regions and are substantially influenced by conventional medical practices and clinician preferences.

Estimates suggest that up to 10% of pregnancies globally are affected by GDM. The prevalence of GDM has markedly increased over the past decade. Additionally, women who have experienced gestational diabetes face a ten-fold higher risk of developing type 2 diabetes later in life. Further, GDM poses risks for the baby, including excessive birth weight; early (preterm) birth; stillbirth; serious breathing difficulties; low blood sugar (hypoglycemia), and a significantly increased risk of obesity and type 2 diabetes later in life.

While the pathogenesis of the disease remains largely unknown, GDM is believed to be a result of interactions between genetic, epigenetic, microbiome, and environmental factors. A variety of risk factors, such as body mass index (BMI) and advancing maternal age, have been associated with increased risk of GDM as well as other pregnancy complications. However, in many cases, GDM occurs in healthy nulliparous women with no obvious risk factors.

Multiple studies suggest that of chief importance among modifiable risk factors are physical activity and dietary intake before conception and during early pregnancy. Reduced level of physical activity during pregnancy is partly responsible for the pregnancy-associated decline in metabolic health.

Since modifying diet and lifestyle are key targets in the prevention and treatment of GDM, it is important to identify women who have a higher risk of developing this pregnancy complication based on genetics, epigenetics, and other factors. Further, it is important to offer to such identified women actionable nutritional and lifestyle recommendations to minimize the risks. It is critical to identify these women either before or during the first trimester of their pregnancy to offer them close monitoring by a healthcare professional.

Studies have shown that the actual implementation of lifestyle modifications reduces the risk of GDM. A systematic review has suggested that lifestyle intervention before the 15th gestational week may reduce GDM by 20%. A randomized controlled trial demonstrated that moderate individualized lifestyle intervention reduced the incidence of GDM by 39% in high-risk pregnant women (Reference 1).

Emerging data suggest that the tendency to develop pregnancy complications has genetic and epigenetic components. Earlier studies have explored a limited number of genes involved in the molecular mechanisms of GDM. It is essential to use large-scale genomics data to identify genetic variations associated with the risk of GDM, which is a complex, and likely heterogeneous pregnancy disorder. Our earlier paper addresses the development of a predictive polygenic risk score for GDM based on genetic variations (Ref. 2). Material in our earlier paper is also documented in U.S. Non-Provisional patent application Ser. No. 18/073,551 entitled “System And Method For Assessing Risk Predisposition To Gestational Diabetes And Developing Personalized Nutrition Plans For Use During Stages Of Preconception, Pregnancy, And Lactation/Postpartum” filed Dec. 1, 2022, the contents of which are incorporated herein in their entirety.

Further, several studies emphasize the significance of DNA methylation in the underlying biological processes of GDM. One study identified potential diagnostic CpG biomarkers in patients with GDM by the combination of an epigenome-wide association study (EWAS) and machine learning model (Ref. 3). Another study identified five CpGs as potential clinical biomarkers for early detection of GDM and therapeutic intervention (Ref. 4). However, the results were not replicated in other cohorts.

Another study found potential methylation biomarkers for GDM in maternal peripheral blood samples through pregnancy as well as candidate genes involved in GDM development (Ref. 5). In this study, EWAS was conducted in 32 pregnant women (16 with GDM and 16 non-GDM) at pregnancy weeks 24-28 and 36-38, and further validated in a larger independent cohort with different ethnic origins. The study identified 272 CpGs that are significantly different between GDM and non-GDM pregnant women across two time points during pregnancy.

The significant CpG sites were related to pathways associated with type 1 diabetes mellitus, insulin resistance, and secretion. However, the CpGs identified in this study were measured in the second and third trimesters of pregnancy. Hence, it was not conclusive whether CpGs correlating with instances of GDM are consequences of GDM, or early drivers of GDM. The studies described above demonstrate the important role that DNA methylation plays in the development of GDM. However, these studies vary greatly in terms of methods, such as study design, execution, and data presentation. Further, CpG methylation markers identified in these studies are not replicated.

Developing a standardized approach for analyzing large-scale DNA methylation repositories is crucial to gaining further insights into the role of DNA methylation in the development of GDM. To this end, a specific embodiment of the present disclosure involves a genomics data repository that contains integrated methylation data and genetics data utilized to develop accurate risk predisposition scores of GDM.

Further, database repositories with genomics data are integrated with non-genomics repositories that contain wearables data and survey data on GDM. As data repositories grow, risk score assessments based on DNA methylation data will be updated by comparing cases (pregnancies with GDM) with controls using machine learning methodologies, and other computational methodologies. The risk predisposition can further be integrated into clinical practice for early identification of women with high risk.

It is therefore crucial to identify women at higher risk of GDM based on genomics data and other factors and provide those women with actionable nutritional and lifestyle recommendations to reduce risks. Ideally, identification of women at higher risk of GDM would occur either at the preconception stage or during the first trimester of pregnancy.

There is hence a large unmet need for systems, methods, and devices that are capable of accurately predicting risk of GDM based on early and modifiable biological markers, such as DNA methylation markers (CpGs) and other available information.

### PRIOR ART

One disclosure to date addresses assessing risks of GDM based on DNA methylation markers. Chinese disclosure CN117187381A filed in late 2023 and entitled “Methylation region marker combination for early-stage auxiliary diagnosis of gestational diabetes mellitus and application thereof” describes the use of seven DNA methylation regions as part of an early auxiliary diagnosis kit for GDM. This disclosure is an attempt for an early diagnosis of GDM but the list of DNA methylation markers (CpGs) the disclosure provides is not exhaustive. It is also unclear whether the measured CpGs in CN117187381A are causally linked to GDM or the consequences of CpGs. Additionally, DNA methylation data cited in CN117187381A is not integrated with wearables data or survey/feedback data. Chinese disclosure CN117187381A does not furnish a platform for collecting genomics and non-genomics data. There are hence shortcomings in CN117187381A regarding assessment of GDM risks.

### BRIEF DESCRIPTION OF THE FIGURES

FIG. 1 is a diagram illustrating modules of a system according to an embodiment of the present disclosure.

FIG. 2 is a block diagram of a system according to an embodiment of the present disclosure.

FIG. 3 is a chart listing CpG markers significantly associated with GDM according to an embodiment of the present disclosure.

### DETAILED DESCRIPTION

Systems and methods provided herein are directed to identifying women at early stages of elevated risk of GDM to allow for early monitoring and to personalize dietary recommendations and lifestyle changes to reduce the risks of this common pregnancy complication. Systems and methods are provided herein for risk predisposition assessment based on DNA methylation data, wearables data, and survey data to produce more accurate assessments, stratify population risks, and identify actionable and modifiable methylation markers. Disclosed systems and methods rectify deficiencies in prior implementations by introducing a dynamic self-learning system for deducing DNA methylation markers (CpGs) causally linked to GDM.

Systems and methods further construct an accurate predictor for GDM risk utilizing a machine learning model. This model undergoes training and validation processes on constantly updated methylation data. The processes are integrated with self-reported information and data collected from wearables.

Systems and methods disclosed herein assess DNA methylation markers for individual women. These markers are integrated with data from wearables and with self-reported information obtained through surveys and feedback mechanisms. Employing at least machine learning methodology, systems and methods predict risk predisposition for GDM in subject individuals. Systems and methods are provided for identification of DNA methylation markers causally linked to GDM.

By leveraging methylation data, data from wearables, and self-reported information, the present disclosure predicts risks of GDM in individual women. A principal objective herein is to enhance evaluation of GDM risk and reveal methylation markers that serve as early and modifiable biomarkers of GDM.

A platform provided herein collects large amounts of heterogeneous data from individuals. The collected data may provide bases for longitudinal studies of GDM and other pregnancy complications and pregnancy-related phenotypes. In embodiments, the platform provides personalized nutrition advice and lifestyle modifications in the stages of preconception and early pregnancy. The advice is tailored to an individual woman's DNA methylation data, genetics data, and other considerations that may be critical to ensure the health and wellness of mothers and babies.

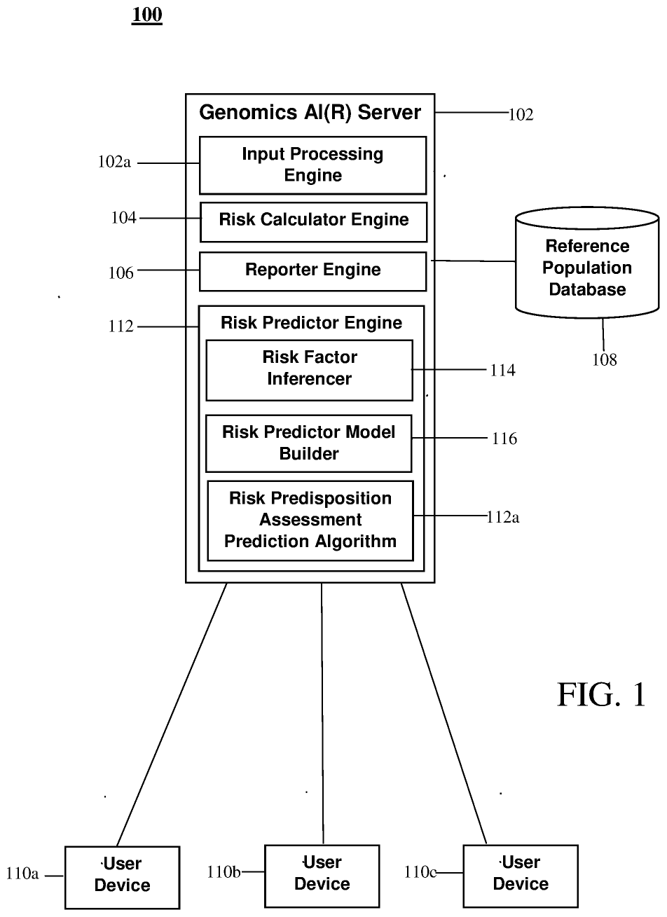

Turning to the figures, FIG. 1 illustrates components and interactions of a system 100 for assessing risk predisposition to GDM. FIG. 1 depicts the system 100 comprising a Genomics AI(R) server 102, or server 102 for brevity. The server 102 comprises an input processing engine 102a, a risk calculator engine 104, a reporter engine 106, a risk predictor engine 112, and a reference population database 108.

The system 100 also comprises a plurality of user devices 110a-c used by individuals to submit data via the input processing engine 102a to the Genomics AI(R) server 102 and to receive personalized reports and other data from the Genomics AI(R) server 102 via the reporter engine 106 and other components. The risk predictor engine 112 comprises a risk factor inferencer 114, a risk predictor model builder 116, and a risk predisposition assessment prediction algorithm 112a. While quantity three user devices 110a-c are depicted in FIG. 1 and provided by the system 100, in embodiments more than or less than quantity three user devices 110a-c may be provided.

The Genomics AI(R) server 102 may be a single computer or multiple physical computers situated at one or multiple geographic locations. The input processing engine 102a, the risk calculator engine 104, the reporter engine 106, and the risk predictor engine 112 are depicted in FIG. 1 as contained by or components of the Genomics AI(R) server 102 and executing on the Genomics AI(R) server 102, but in embodiments these components may be separate components or software executing on separate devices proximate or remote from the Genomics AI(R) server 102.

The system 100 also comprises, as noted, the risk predisposition assessment prediction algorithm 112a that is a component of the risk predictor engine 112. In some embodiments, the predisposition assessment prediction algorithm 112a may not be a component of the risk predictor engine 112 and may instead execute independently and not on the genomics AI(R) server 102. It may comprise more than one algorithm as well.

Use of the term “application” herein may refer to various components of the system 100 described herein as well as the system 200 described below. The term may in embodiments refer to software components, hardware components, or a combination of hardware and software components.

While referred to as engines, the input processing engine 102a, the risk calculator engine 104, the reporter engine 106, and the risk predictor engine 112 may be combinations of hardware and software applications or entirely software applications. Components described herein as modules, submodules, or devices may be physical devices, combinations of a physical device and software, or entirely software. For example, a risk factor inferencer module 114 and a risk model builder module 116 may be combinations of hardware and software or primarily software.

Methylation data, data quantified from wearables, and/or screening tests are received by the Genomics AI(R) server 102. In addition, self-reported data from individuals using user devices 110a-c is also received by the server 102. The received material is processed by the input processing device 102a and stored at least in the reference population database 108.

The received data is also provided to the risk calculator engine 104 to compute a risk predisposition to GDM for an individual by applying algorithms comprising at least a risk predisposition assessment prediction algorithm 112a to at least the received data. The risk predictor engine 112 is also applied to compute risk predisposition to GDMs.

Based on risk of GDM calculated by the risk calculator engine 104 and other components, the reporter engine 106 generates a personalized report for the subject individual with a predicted risk predisposition to GDM based on methylation markers. In an embodiment, the risk predisposition to GDM is based on methylation markers (CpGs) causally linked to GDM. These CpGs are identified in biological samples of the reference population by the Mendelian Randomization methodology. The personalized report may further contain actionable nutrition and lifestyle plans tailored to the individual woman.

The personalized report may further contain comparisons of the subject individual's data with the reference population data and contain comparisons of the individual's data at different times. Personalized reports may further be utilized by the individual, or third parties, for example, healthcare professionals, for recommending comprehensive monitoring and/or preventative nutrition and lifestyle programs to mitigate the risks.

Feedback may be provided that leads to collection of data at later times via survey questionnaires, and/or quantified data from wearables, or screening tests. Collected data is sent back to the reporter engine 106 and the reference population database 108. Additional methylation data may later be collected from the individual and transmitted to the reference population database 108. Data collected at least via feedback is utilized to build a longitudinal data platform for improving the risk predisposition prediction to GDM and identifying methylation markers causally linked to GDM.

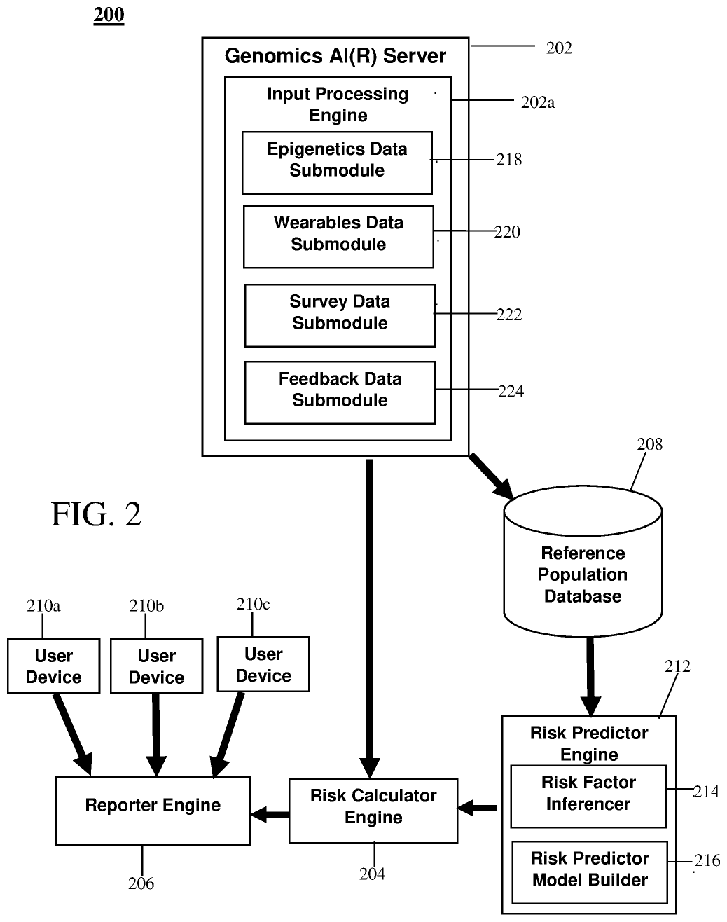

FIG. 2 is a block diagram of a system for assessing risk predisposition to GDM based on methylation markers, wearables, and survey data according to an embodiment of the present disclosure. FIG. 2 depicts components and interactions of a system 200 in which components are indexed to components of the system 100 described above.

System 200 comprises a genomics AI(R) server 202, an input processing device 202a, a risk calculator engine 204, a reporter engine 206, and a reference population database 208. System 200 also comprises user devices 210a-c, a risk predictor engine 212, a risk factor influencer 214, and a risk predictor model builder 216.

The input processing engine 202a receives epigenetics data, and other information from a subject via user devices 210a-c. The input processing engine 202a consists of four submodules: an epigenetics data submodule 218, a wearables data submodule 220, a survey data submodule 222, and a feedback data submodule 224. In some embodiments, data input is provided via a web, or mobile application at home, or in a professional environment at a healthcare provider.

The input processing engine 202a receives and processes methylation data from various sources via the epigenetics data submodule 218 which may be integrated with external information providers or databases. In some embodiments, methylation data may be a file that contains DNA methylation markers (CpGs) uploaded by an individual, uploaded by an external genotyping or sequencing service/company using a generic or proprietary application programming interface (API), or uploaded by a third party, for example, healthcare provider. In embodiments, DNA methylation markers (CpGs) are pre-processed using appropriate bioinformatics methods directed to obtaining quantifiable results to enable further assessments.

The input processing engine 202a receives and processes data from wearables via the wearables data submodule 220. Wearables data may come from biosensors such as wearable ECG Monitors, blood pressure monitors, pulse oximeters, smartwatches with health features, temperature-tracking wearables, sleep trackers, fitness trackers, smart rings, or smart clothing for health monitoring.

The wearables data submodule 220, which may be partially integrated with external information providers, enables input of quantified data by generic or proprietary API. This data may be provided by sensors, wearables, and other relevant devices that report results of screening health tests or from third-party expert reports, for example, from physicians, healthcare providers, wellness coaches.

The input processing engine 202a receives survey data from various sources via the survey data submodule 222. Survey data includes at least chronological age and may include a woman's ethnicity, preconception/pregnancy/postpartum stage, demographics, height, weight, activity level, diet, habits, lifestyle, medical history, geolocation, environment, and preferences. The survey data submodule 222 enables integration with self-reported questionnaires or data input by third parties.

The feedback data submodule 224 is utilized when a woman provides feedback regarding a personalized report. The feedback data submodule 224 may receive data from wearables, screening health tests, or self-reported data at the stage of preconception and pregnancy. Self-reported data may contain information on adverse effects during pregnancy such as morning sickness, nausea, weight gain during pregnancy or weight loss postpartum, blood pressure, pregnancy complications, baby gestational age, baby weight, and lactation issues.

In some embodiments, the feedback data submodule 224 enables input of methylation data to compare the methylation of an individual woman before and after a recommended nutrition or lifestyle plan. The feedback data submodule 224 also receives reviews, survey responses, or other feedback from the individual about specific recipes, food recommendations, and likes/dislikes. The feedback data submodule 224 may be used by the user, or a third party, for example, a healthcare professional, to report adverse reactions such as morning sickness or nausea to specific foods or recipes.

Upon receipt of at least one of methylation data, wearables data, and survey data, the input processing engine 202a propagates the received data to the reference population database 208 which is a repository of methylation, wearables data, and survey data for a plurality of individuals. Material stored in the reference population database 208 is updated with new entries received from individuals via the input processing engine 202a. The reference population database 208 can also be updated by bulk downloads of methylation data and other material from multiple individuals and from public repositories of methylation data and other material, as well as wearables data from external sources, data repositories, and third parties.

Feedback data, received from the user or third-party, is propagated to the reference population database 208. After processing, using suitable data analysis tools, feedback data is further propagated to the risk predictor engine 212 and reporter engine 206 to further improve algorithms including the risk predisposition assessment prediction algorithm 112a provided by the system 100, and identify methylation markers that may be causal drivers of GDMs or causal protectors from GDMs.

A continuous self-learning system may thereby be set into place. For example, by analyzing, via the risk predictor engine 212, collected data in the reference population database 208, the system 200 improves risk predictions for GDMs. The system 200 may further build predictive models for other pregnancy-related complications, including gestational hypertensive disorders.

The system 200 may infer, by analyzing via computational algorithms and collected data, that women with specific combinations of methylation markers are more likely to have morning sickness in the first trimester if they consume specific foods. Similarly, the system 200 may learn that specific foods and recipes help women deal with morning sickness and nausea.

The system 200 may infer, by analyzing via computational algorithms and collected data, that specific nutritional interventions or lifestyle changes, affect methylation markers related to GDMs. These nutritional and lifestyle changes will therefore be utilized in the updated reports.

The reference population database 208 provides a basis for updating, via a machine learning methodology, the risk predictor engine 212. Further, the reference population database 208 may provide a basis for generating a personalized report performed by the reporter engine 206.

The risk predictor engine 212 comprises a risk factor inferencer 214 and a risk predictor model builder 216. The risk factor inferencer 214 identifies, by applying at least epigenome-wide Mendelian Randomization (EWMR), methylation markers causal to GDMs. The risk factor inferencer 214 further validates identified methylation markers using the data from the reference population database 208.

Mendelian randomization (MR) is an established genetic computational approach for causal inference that recapitulates the principle of a randomized clinical trial (RCT) as it utilizes genetic variants as instrumental variables. While RCTs generally consider the effect of treatment (exposure) by comparing the cases and the controls, the MR uses the genetic variants (SNPs) that are robustly associated with the exposure as instrumental variables as SNPs are randomly assigned at conception and therefore are not biased by environmental confounders. Hence, MR is used as a computational tool for investigating causal relationships between DNA methylation, as exposure, and GDM as an outcome.

In one embodiment, epigenome-wide Mendelian Randomization (EWMR) employs summary statistics data from a genome-wide association study (GWAS) on GDM, sourced from the MR-Base GWAS catalog (Ref. 6), as the outcome. In a specific instance, the outcome is based on gestational diabetes data from the Finnish Gestational Diabetes study (Ref. 7). Additionally, EWMR utilizes a publicly available dataset with 11,165,559 SNP-CpG associations (meQTLs; P<10-14, whole blood samples) identified through GWAS from 6994 samples (Ref. 8) as exposures.

In the embodiment introduced immediately above, the EWMR yields 497 CpGs causally contributing to GDM (p<=0.01). CpGs that are causally linked to increased risk of GDM are driver CpGs, and CpGs that are causally linked to lower risk of GDM are protector CpGs.

These candidate causal CpGs, and genes that annotate them, are significantly (p<0.001) enriched in multiple processes related to the development of GDM. Specifically, genes that annotate these CpGs are enriched in glucose regulation processes, including insulin glucose pathway, levels of fasting glucose, glucose import, and its regulation. Further, genes with roles in the development of Type 2 diabetes are over-represented in the list of genes that annotate causal CpGs.

The causal driver CpGs are significantly enriched in adhesion-related processes and pathways. Functional analyses on the gene and associated protein levels support these findings. This is in line with an epigenome-wide association study (EWAS) (Ref. 5) that identified the most differentially methylated site in the SELP gene by comparing the GDM group of pregnant women to the control group. The SELP gene encodes for P-Selectin, a granular membrane protein and a cellular adhesion molecule that mediates the interaction of activated endothelial cells or platelets with leukocytes.

Further, CpGs causally linked to GDM are enriched in endothelial cell morphogenesis and its regulation. Extending the analysis to the protein space via the EWAS catalog identifies several related proteins. For example, proteins correlated with GDM incidences are enriched in endothelial cell migration and endothelial growth factor stimulus, while proteins that are anti-correlated with GDM incidences are enriched in endothelial cell proliferation.

Also, several immune-related processes, such as type 2 immune response, regulation of immune response, T cell proliferation, B cell differentiation, production of interferons and interleukins, and inflammatory responses are significantly over-represented in CpGs causally linked to GDM. This is in line with other studies. For example, epigenome-wide and transcriptome-wide analyses reveal that gestational diabetes is associated with over-representation of immune response pathways, reflecting these coordinated changes in the MHC region (Ref. 9).

Further, embryonic placenta morphogenesis is significantly (p-value=0.0002) over-represented in CpGs causal to GDM.

These findings demonstrate that CpG sites causally linked to GDM can be identified from the whole blood data and may assist in elucidating biological mechanisms underlying GDM and providing clues for the discovery of drug targets.

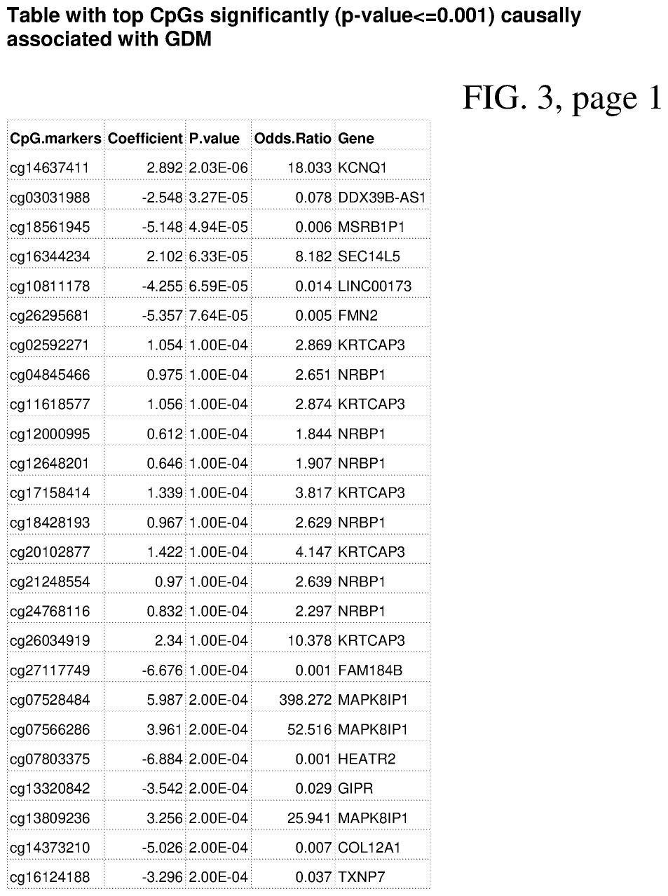

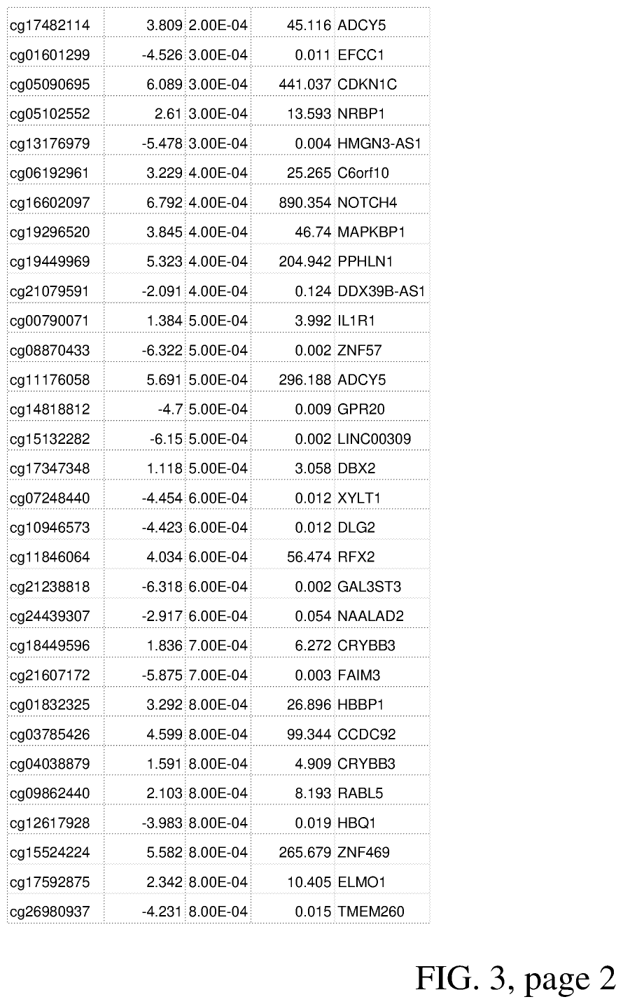

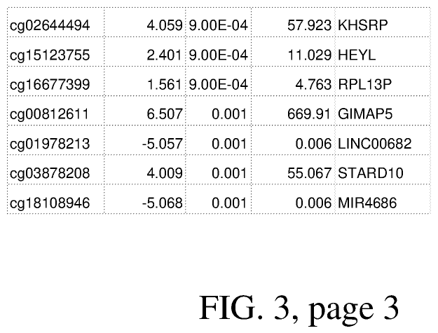

FIG. 3 is a chart listing CpG markers significantly (p-value<=0.001) associated with GDM according to an embodiment of the present disclosure. There are 40 driver CpGs and 23 protector CpGs.

In alternative embodiments, different meQTL datasets, whether publicly available or proprietary, may be employed as exposures in EWMR. An illustration of this is the GoDMC meQTL dataset, which encompasses SNP-CpG associations for 420,509 CpG sites identified in whole blood samples from 27,750 subjects. In other embodiments, pregnancy complications such as gestational hypertension, preeclampsia, adverse cardiac events, and pregnancy-related phenotypes such as morning sickness, nausea, weight gain during pregnancy or weight loss postpartum, baby gestational age, baby weight, and lactation issues can be used as outcomes in the EWMR analyses to infer methylation markers causal to these pregnancy complications.

In certain embodiments, Mendelian Randomization takes the form of a two-sample Mendelian Randomization utilizing linear regression. Alternatively, in specific instances, it adopts a three-sample Mendelian Randomization approach. Furthermore, in other embodiments, Mendelian Randomization employs non-linear models to depict the relationship between exposure and outcome. Those skilled in the art understand that various models, both linear and nonlinear, can be constructed between exposures and outcomes to deduce causal methylation markers.

The risk prediction model builder 116 develops, employing a supervised machine learning methodology, a predictive model for assessing the risk of GDM by leveraging data from the reference population storage 108. In the preferred embodiment, CpGs causally linked to GDM are utilized as input features. The risk prediction model builder 216 may conduct computations for predictive risk models using at least one algorithm, which could be proprietary and/or developed by a third-party source while adhering to the best practices of machine learning.

The risk predictor model builder 216 develops a methylation risk score (MRS) predicting the present risk of GDM. The MRS model is constructed using a machine learning methodology, utilizing a training dataset comprising biological samples of cases (women with GDM) and controls (women who did not have GDM). Each biological sample includes methylation markers measured through external genotyping or sequencing services. In preferred embodiments, biological samples are whole blood samples or blood plasma samples. In other embodiments, biological samples are saliva. In a preferred embodiment, MRS is a risk-weighted linear sum of methylation levels at multiple CpG sites (Ref. 10).

The validation of the risk predictor is carried out on an independent dataset of biological samples. In preferred embodiments, both the training and validation datasets incorporate proprietary data. Publicly available datasets are integrated with proprietary data, subject to pre-processing and normalization through bioinformatics methods. The datasets are then partitioned into training and validation subsets to ensure at least age balance.

In the preferred embodiment, the risk predictor model is learned using an elastic net model using, as input features, CpG markers causal to GDM as identified by EWMR in the risk inference module. In other embodiments, the risk predictor is learned using an elastic net model using, as features, CpG markers that are significantly associated with GDM, and other relevant markers extracted from wearables data, screening tests, or survey data.

In a specific embodiment, the elastic net model includes the CpG feature-specific penalty factor informed by the causality rank that is based on at least one of the causal effect sizes and p-values from EWMR analyses, or a quantitative measure computed by a bioinformatics tool (e.g. colocalization probability). In other embodiments, the risk predictor is learned by another supervised machine learning algorithm, wherein CpG features are ranked by taking the causality rank into account. In another embodiment, CpG features are ranked by a quantitative measure based on correlations with hypertension and other markers extracted from wearables data, screening tests, or survey data.

The risk calculator engine 204 is a computing device that receives methylation data from an individual via the input processing engine 202a. It further receives causal methylation markers and the risk predictor model from the risk predictor engine 212. The risk calculator engine 204 further identifies causal methylation markers in the individual's methylation data and calculates the risk predisposition to GDM of the individual using the risk predictor model.

The reporter engine 206 receives causal methylation markers and risk predisposition to GDM for an individual from the risk calculator engine 204 and generates a personalized report informing individuals about their risk predisposition to GDM, methylation markers that contribute to the risk, and methylation markers that protect from the risk. In other embodiments, specific foods and recipes can be identified, by computational analyses, as reversing the effect of specific damaging methylation markers or improving protective methylation markers.

Feedback provided by individuals is done via user devices 210a-c that collect data and responses from individuals on the personalized reports provided by the system. The feedback data may comprise methylation data collected from individuals at different time points. Feedback data may further comprise wearables data and data from survey questionnaires. Feedback responses may comprise questionnaires on comprehension of information provided by personalized reports.

Feedback responses may comprise liking/disliking recommendations of foods and recipes. Feedback data may then be transmitted to the reference population database 208 by using the feedback data submodule 224, and, after processing, be further transmitted to the risk predictor engine 212 to improve risk prediction algorithms. A continuous self-learning system may thereby be set in place. The feedback responses may be transmitted to the reporter engine 206 to improve personalized reports.

In some embodiments, the user devices 210 a-c may be mobile computing devices such as a smartphone or a tablet computing device. In some embodiments, the user devices 210 a-c may be a desktop computing device or a laptop computing device. In some embodiments, the user devices 210 a-c may include more than one computing device, such as a user computing device configured to provide a user interface and one or more server computing devices configured to provide computational functionality. In such embodiments, the user computing device and one or more server computing devices may communicate via any suitable communication technology or technologies, such as a wired technology (including but not limited to Ethernet, USB, or the Internet) or wireless technology (including but not limited to WiFi, WiMAX, 3G, 4G, LTE, or Bluetooth).

The steps of systems and methods provided herein may be as follows:
1. The system receives an individual person's epigenetic data (DNA methylation markers, CpGs).
2. The system adds the individual's methylation data to the population's methylation data and compares the individual's methylation data to the reference population data.
3. The system further receives wearable data of the individual, via a wearable device, or sensor.
4. The system adds the individual's wearables data to population wearables data, compares the individual's wearables data to the reference population wearables data, and integrates population wearables data with population methylation data.
5. The system further receives survey data from the individual woman, which can be self-reported, or collected by a healthcare provider. Survey data includes at least an individual's age, and it may further include information on general health, diet, and lifestyle data.
6. The system adds the individual woman's survey data to the reference population survey data, compares the individual woman's survey data to the reference population survey data, and integrates population methylation data with population wearables data and population survey data.
7. The system collects longitudinal data that includes methylation data, wearables data, and survey data from a plurality of individuals measured at various time intervals. Survey data may include feedback on food and recipe recommendations, including liking/disliking, subjective assessments, and adverse effects. Survey data may be self-reported or reported by a third party.
8. The system propagates the individual woman's longitudinal data to storage with population data.
9. The system calculates the risk predisposition to GDM for the individual woman by employing a risk predictor model that utilizes multiple methylation markers identified in the methylation data specific to that individual woman.
10. The system generates a personalized report that contains the individual woman's predicted risk predisposition to GDM, related methylation markers, measurements from wearables data, and relevant markers from screening tests.
11. The system relies on a reporting and feedback module to send and receive the material.

### REFERENCES

1. Altemani and Alzaheb. The prevention of gestational diabetes mellitus (The role of lifestyle): a meta-analysis. Diabetol Metab Syndr. 2022 Jun. 15; 14(1):83. doi: 10.1186/s13098-022-00854-5. PMID: 35706048; PMCID: PMC9199329.
2. Perišić et al. Polygenic Risk Score and Risk Factors for Gestational Diabetes. J Pers Med. 2022 Aug. 26; 12(9):1381. doi: 10.3390/jpm12091381. PMID: 36143166; PMCID: PMC9505112.
3. Liu et al. Identification of Diagnostic CpG Signatures in Patients with Gestational Diabetes Mellitus via Epigenome-Wide Association Study Integrated with Machine Learning. Biomed Res Int. 2021 May 19; 2021:1984690. doi: 10.1155/2021/1984690. PMID: 34104645; PMCID: PMC8162250.
4. Wu et al. Maternal genome-wide DNA methylation profiling in gestational diabetes shows distinctive disease-associated changes relative to matched healthy pregnancies. Epigenetics. 2018; 13(2):122-128. doi: 10.1080/15592294.2016.1166321. Epub 2018 Jan. 25. PMID: 27019060; PMCID: PMC5873366.
5. Linares-Pineda et al. Epigenetic marks associated with gestational diabetes mellitus across two time points during pregnancy. Clin Epigenetics. 2023 Jul. 6; 15(1):110. doi: 10.1186/s13148-023-01523-8. PMID: 37415231; PMCID: PMC10324212.
6. Hemani et al. The MR-Base platform supports systematic causal inference across the human phenome. eLife. 2018; 7:e34408. doi: 10.7554/eLife.34408. PMID: 29846171; PMCID: PMC5976434.
7. Keikkala et al. Cohort Profile: The Finnish Gestational Diabetes (FinnGeDi) Study. Int J Epidemiol. 2020 Jun. 1; 49(3):762-763g. doi: 10.1093/ije/dyaa039. PMID: 32374401; PMCID: PMC7394962.
8. Hawe et al (2022) Genetic variation influencing DNA methylation provides insights into molecular mechanisms regulating genomic function. Nat Genet. 54(1):18-29.
9. Binder et al. Epigenome-wide and transcriptome-wide analyses reveal gestational diabetes is associated with alterations in the human leukocyte antigen complex. Clin Epigenetics. 2015 Aug. 5; 7(1):79. doi: 10.1186/s13148-015-0116-y. PMID: 26244062; PMCID: PMC4524439.
10. Thompson et al. Methylation risk scores are associated with a collection of phenotypes within electronic health record systems. NPJ Genom Med. 2022 Aug. 25; 7(1):50. doi: 10.1038/s41525-022-00320-1. PMID: 36008412; PMCID: PMC9411568.

What is claimed is:

1. A method for computing predisposition risk for gestational diabetes mellitus (GDM) of an individual female based at least on methylation, comprising:
receiving, by a computing device, methylation data including methylation markers for a female human;
receiving, by the computing device, wearables data for the female;
receiving, by the computing device, survey data provided by the female;
applying, by the computing device, a risk predisposition predictor model to at least the received data to compute a risk predisposition to gestational diabetes mellitus of the female.

2. The method of claim 1, further comprising the computer identifying methylation markers (CpGs) causally linked to gestational diabetes mellitus in the methylation data.

3. The method of claim 1, further comprising the computer generating a personalized report for the individual, the report describing the computed predisposition risk assessment based at least on methylation markers, and the report further containing risk factors identified in wearables data and describing methylation markers causally linked to gestational diabetes mellitus, the markers identified at least in the received data.

4. The method of claim 3, further comprising the computing device providing a self-learning system for deducing DNA methylation markers (CpGs) causally linked to GDM and further revealing methylation markers that serve as early and modifiable biomarkers of GDM.

5. The method of claim 1, further comprising the computer copying the received data and the computed predisposition risk for gestational diabetes mellitus to a reference population database.

6. The method of claim 1, wherein the risk predisposition predictor model is trained and validated on reference population data stored in the reference population database.

7. The method of claim 1, wherein wearables data is generated by at least biosensors comprising at least one of wearable glucose monitoring, ECG monitors, blood pressure monitors, pulse oximeters, smartwatches with health features, temperature-tracking wearables, sleep trackers, fitness trackers, smart rings, and smart clothing for health monitoring.

8. The method of claim 7, wherein risk factors of gestational diabetes mellitus are extracted, via a machine learning (AI) classifier, from the wearables data, wherein the classifier is one of a proprietary, open-source, and a third-party algorithm utilized via an application programming interface (API).

9. A system for continual improvement of risk predisposition assessment to gestational diabetes mellitus (GDM) based at least on methylation data, comprising:
a computer and application executing thereon that:
receives epigenetics data containing at least DNA methylation markers describing an individual female,
receives wearables data describing the female,
receives feedback data and survey data comprising at least a chronological age of the female, and
computes, based at least on accessing a risk predisposition assessment prediction algorithm, a risk predisposition assessment to gestational diabetes mellitus of the female based at least on the data.

10. The system of claim 9, wherein risk predisposition to GDM is based at least on methylation markers (CpGs) causally linked to GDM and wherein the CpGs are identified in biological samples of reference populations by at least Mendelian Randomization methodology.

11. The system of claim 9, wherein the feedback data is further propagated to a risk predisposition assessment predictor engine and a reporter engine to improve the risk predisposition assessment prediction algorithm and identify methylation markers that either contribute to the risk of gestational diabetes mellitus (causal drivers) or causal protector from the risk of gestational diabetes mellitus (causal protectors).

12. The system of claim 9, wherein DNA methylation markers (CpGs) are pre-processed using bioinformatics methods directed to obtaining quantifiable results aiming to acquire quantifiable results for subsequent assessments.

13. The system of claim 9, wherein the system enables input of methylation data to compare risk predisposition to gestational diabetes mellitus of individuals before and after recommended nutritional and lifestyle programs.

14. The system of claim 9, wherein the system builds predictive models for risk predisposition assessments to pregnancy-related or postpartum-related phenotypes comprising at least one of gestational diabetes, cardiac complications, preterm birth, morning sickness, nausea, and postpartum depression.

15. A method for using methylation markers associated with pregnancy-related phenotypes, comprising:
a computer applying epigenome-wide Mendelian Randomization (EWMR) to received data describing at least one individual female;
the computer identifying, via the applied EWMR, methylation markers (CpGs) causal to at least one pregnancy-related phenotype;
the computer utilizing epigenome-wide methylation (meQTL) data as exposure; and
the computer validating the identified methylation markers.

16. The method of claim 15, further comprising the computer validating methylation markers using methylation data from a reference population database.

17. The method of claim 15, further comprising the computer applying the EWMR to utilize summary statistics from genome-wide association studies for pregnancy-related phenotypes as outcomes.

18. The method of claim 15, further comprising the computer observing and measuring risk factors from at least one of wearables data, survey, and feedback data.

19. The method of claim 15, wherein epigenome-wide methylation data (meQTL) contain SNP-CpG associations detected in a plurality of biological samples comprising at least one of whole blood, blood plasma, and saliva.

20. The method of claim 15, wherein methylation markers (CpGs) associated with at least one pregnancy-related phenotype are identified by at least one of the correlative analyses and generalized linear regression from reference population data and wherein pregnancy-related phenotype data are at least one of observable and measurable and are extracted from at least one of pregnancy-related phenotype data, wearables data, survey data, and feedback data.
